(19) United States
(12) Patent Application Publication
(10) Pub. No.: US 2021/0322597 A1
(43) Pub. Date: Oct. 21, 2021

(54) UV DISINFECTION SYSTEM

(71) Applicant: ENERGY FOCUS, INC., Solon, OH (US)

(72) Inventors: James Tu, Englewood Cliffs, NJ (US); Christopher F. Prochaska, Aurora, OH (US); Greg Flies, Chagrin Falls, OH (US); David Nicolay, Fairview Park, OH (US); John M. Davenport, Tucson, AZ (US)

(21) Appl. No.: 17/232,443

(22) Filed: Apr. 16, 2021

Related U.S. Application Data
(60) Provisional application No. 63/011,846, filed on Apr. 17, 2020.
(60) Provisional application No. 63/090,446, filed on Oct. 12, 2020.

(51) Int. Cl. A61L 2/10; H05B 47/115; H05B 45/10; H05B 47/16; A61L 9/20

(52) U.S. Cl. CPC A61L 2/10; H05B 47/115; H05B 45/10; H05B 47/16; A61L 2209/12; A61L 2202/11; A61L 2202/14; A61L 2209/10; A61L 9/20

(57) ABSTRACT

According to one aspect, a system for ultraviolet (UV) disinfection may include a switch and a lamp fixture. The lamp fixture may include a UV lamp and a UV controller. The switch may be configured to receive a command to engage in a UV disinfection mode. The UV controller may be configured to receive the command to engage in the UV disinfection mode from the switch via a switched line. The UV controller may activate the UV lamp based on the received command to engage in the UV disinfection mode.

### BACKGROUND

Ultraviolet germicidal irradiation (UVGI) is a disinfection method that uses short-wavelength ultraviolet (e.g., ultraviolet or UV) light to kill or inactivate microorganisms by destroying nucleic acids and disrupting their DNA, leaving them unable to perform vital cellular functions. UVGI is used in a variety of applications, such as food, air, and water purification, and may be effective at destroying viruses, such as the COVID-19 virus.

### BRIEF DESCRIPTION

According to one aspect, a system for ultraviolet (UV) disinfection may include a switch and a lamp fixture. The lamp fixture may include a UV lamp and a UV controller. The switch may be configured to receive a command to engage in a UV disinfection mode. The UV controller may be configured to receive the command to engage in the UV disinfection mode from the switch via a switched line. The UV controller may activate the UV lamp based on the received command to engage in the UV disinfection mode.

The UV lamp may be mounted on an exterior of the lamp fixture. The UV lamp may be mounted on an interior of the lamp fixture within an optically enclosed area and the lamp fixture may include a fan configured to blow air into the optically enclosed area. It will be appreciated that the fan described in this application could also be configured to remove air from the optically enclosed area without departing from the scope of this disclosure. The lamp fixture may include an indicator light configured to activate when the lamp fixture is engaged in the UV disinfection mode.

The lamp fixture may include a communication interface configured to receive sensor data from a remote sensor. The UV controller may deactivate the UV lamp based on the data from the remote sensor being indicative of a detected presence of a life form. The UV controller may include a lamp life timer configured to track an active operation time for the UV lamp. The lamp fixture may include an indicator light configured to activate when the active operation time exceeds a predetermined threshold. The UV controller may be configured to receive the command as a placeholder bit via the switched line. The lamp fixture may include a mounted sensor. The UV controller may deactivate the UV lamp based on data from the mounted sensor being indicative of a detected presence of a life form.

According to one aspect, a system for ultraviolet (UV) disinfection may include a switch and a lamp fixture. The lamp fixture may include a first UV lamp, a second UV lamp, a fan, and a UV controller. The switch may be configured to receive a command to engage in a UV disinfection mode. The first UV lamp may be mounted on an exterior of the lamp fixture. The second UV lamp may be mounted on an interior of the lamp fixture within an optically enclosed area. The fan may be configured to blow air into the optically enclosed area. The UV controller may be configured to receive the command to engage in the UV disinfection mode from the switch via a switched line. The UV controller may activate the UV lamp based on the received command to engage in the UV disinfection mode.

The lamp fixture may include an indicator light configured to activate when the lamp fixture is engaged in the UV disinfection mode. The lamp fixture may include a communication interface configured to receive sensor data from a remote sensor. The UV controller may deactivate the first UV lamp based on the data from the remote sensor being indicative of a detected presence of a life form.

According to one aspect, a system for ultraviolet (UV) disinfection may include a switch and a lamp fixture. The lamp fixture may include a UV lamp, a fan, and a UV controller. The switch may be configured to receive a command to engage in a UV disinfection mode. The UV lamp may be mounted on an interior of the lamp fixture within an optically enclosed area. The fan may be configured to blow air into the optically enclosed area. The UV controller may be configured to receive the command to engage in the UV disinfection mode from the switch via a switched line. The UV controller may activate the UV lamp based on the received command to engage in the UV disinfection mode.

The switch may transmit at least one command to the lamp fixture by providing brief interruptions of the power line to the lamp fixture. The lamp fixture may include a decoder which interprets and executes the at least one command. The lamp fixture may include an indicator light configured to activate when the lamp fixture is engaged in the UV disinfection mode. The lamp fixture may include a communication interface configured to receive sensor data from a remote sensor. The UV controller may deactivate the UV lamp based on the data from the remote sensor being indicative of a detected presence of a life form. The UV controller may be configured to receive the command as a placeholder bit via the switched line. The lamp fixture may include a mounted sensor. The UV controller may deactivate the UV lamp based on data from the mounted sensor being indicative of a detected presence of a life form.

### BRIEF DESCRIPTION OF THE DRAWINGS

FIG. 1 is an exemplary component diagram of a switch for driving a light emitting diode (LED) lighting fixture, according to one aspect.

FIG. 2 is an exemplary component diagram of a circuit for light emitting diode (LED) lighting with ultraviolet (UV) disinfection, according to one aspect.

FIG. 3 is an exemplary component diagram of a circuit for light emitting diode (LED) lighting with ultraviolet (UV) disinfection, according to one aspect.

FIG. 4 is an exemplary component diagram of a circuit for light emitting diode (LED) lighting with ultraviolet (UV) disinfection, according to one aspect.

FIG. 5 is an exemplary component diagram of a circuit for light emitting diode (LED) lighting with ultraviolet (UV) disinfection, according to one aspect.

FIG. 6 is an exemplary component diagram of a circuit for light emitting diode (LED) lighting with ultraviolet (UV) disinfection, according to one aspect.

FIG. 7 is an exemplary component diagram of a circuit for light emitting diode (LED) lighting with ultraviolet (UV) disinfection, according to one aspect.

### DETAILED DESCRIPTION

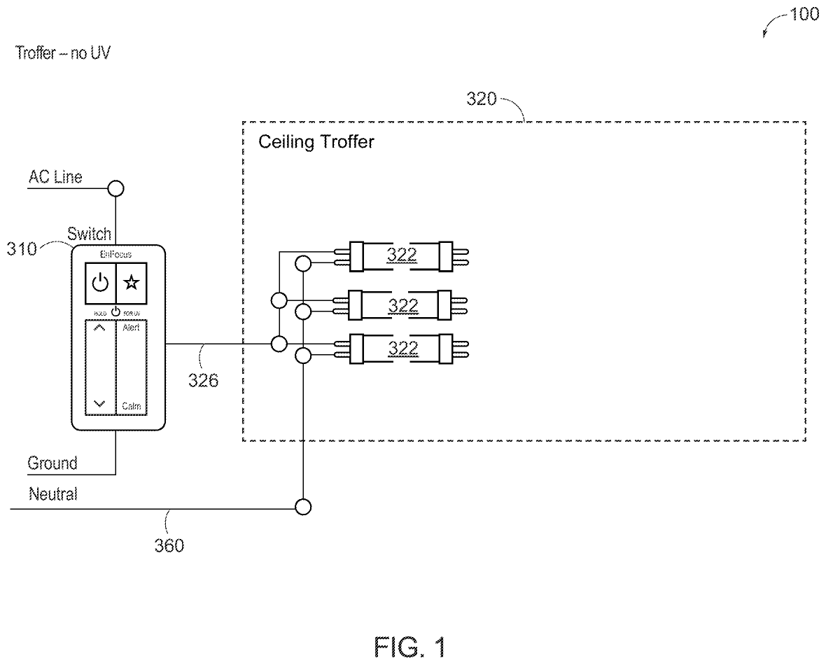

FIG. 1 is an exemplary component diagram of a switch for driving a light emitting diode (LED) lighting fixture, according to one aspect. In FIG. 1, the configuration of the switch is a “two wire” (i.e., no neutral).

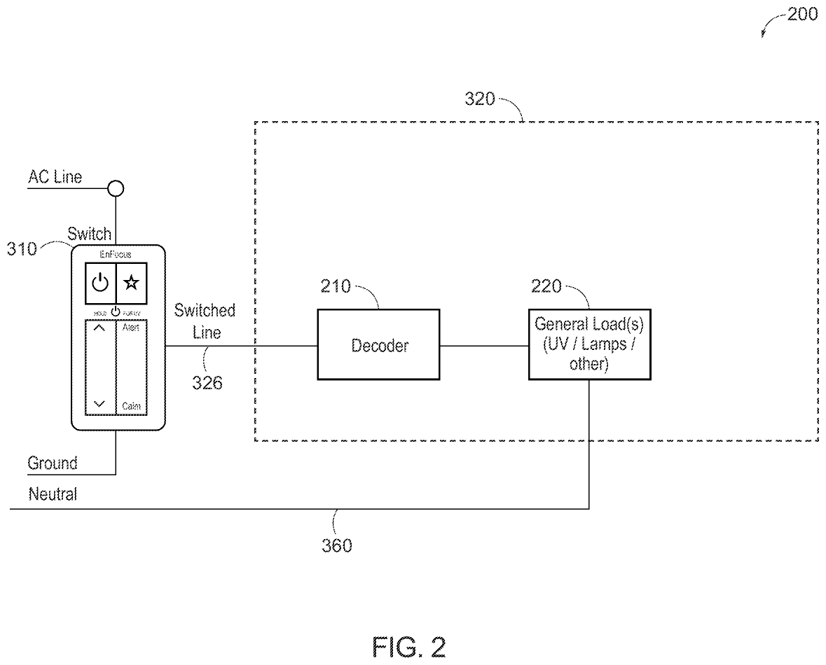

FIG. 2 is an exemplary component diagram of a circuit 200 for light emitting diode (LED) lighting with ultraviolet (UV) disinfection, according to one aspect. In FIG. 2, a lamp fixture decoder 210 performing decoding functions is separate from the general loads 220, which may include UV lamps, LED lamps, etc.

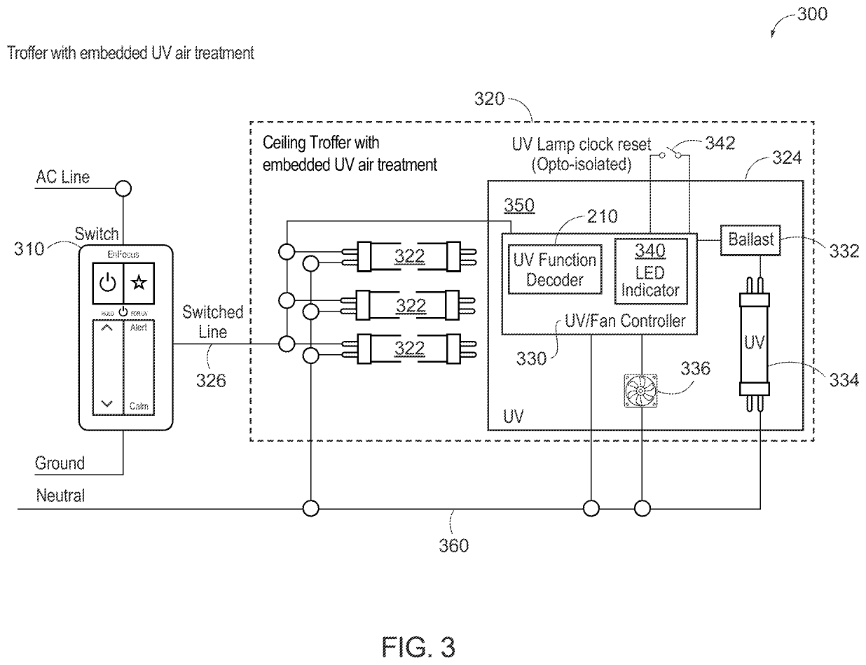

FIG. 3 is an exemplary component diagram of a circuit 300 for light emitting diode (LED) lighting with ultraviolet (UV) disinfection, according to one aspect. As seen in FIG. 3, a system for ultraviolet (UV) disinfection may include a switch 310 and a lamp fixture 320 equipped with one or more LEDs 322 (aka LED lamps) and one or more UV disinfection lamps 334 and a housing 324. As will be appreciated, the LED lamps 322 can be used for general lighting purposes, whereas the UV disinfection lamps 334 can be used for disinfection purposes. Further, the lamps 322, 334 may be replaceable. The LED lamps 322 may be electrically connected to a switched line 326 which is connected to the switch 310. The lamp fixture 320 may include a UV controller 330 which is also connected to the switched line 326, a ballast 332, the UV lamp 334, and a fan 336. The UV controller 330 may include a decoder which may be the decoder 210 described above.

The switch 310 may transmit at least one command to the lamp fixture 320 by providing brief interruptions of the power line to the lamp fixture 320. Thus, the switch 310 can control the LED lamps 322 and also the UV disinfection lamps 334. The switch 310 may include a switch encoder that provide brief interruptions of the line. The lamp fixture decoder 210 may be a line decoder which may interpret brief line interruptions as commands to enable the lamp, disable the lamp, and/or change one or more settings associated with the lamp. The lamp fixture 320 may include the decoder 210 which interprets and executes the at least one command. For example, a “place holder” bit location from data transmitted from the switched line may include UV control information indicative of a command for the UV controller 330 to control the UV lamp 334 and/or fan 336, such as but not limited to turning the UV lamp 334 on, turning the UV lamp 334 off, turning the fan 336 on, turning the fan 336 off, changing a speed of the fan 336, changing a UV level associated with the UV lamp 334, etc. The lamp fixture 320 may include an indicator light 340 configured to activate when the lamp fixture 320 is engaged in the UV disinfection mode.

The UV controller 330 may include a clock reset switch 342 which, when dosed, resets a dock count on the UV lamp usage associated with replacing the UV lamp 334, which may be replaced on a periodic basis. The UV controller 330 may measure and/or track an amount of on time for the UV lamp 334. When a predetermined time (e.g., tracking the amount of on time for the UV lamp 334) has elapsed (e.g., 10,000 hours) one of the LED lamps 322 or the indicator light 340 may be lit to alert a user that it is time to replace the UV lamp.

The clock reset switch 342 may be a single-pull-single-throw (SPST) type switch or other toggle switch. The user may change a state of the clock reset switch 342 (i.e., from open to closed or from closed to open) as part of the UV lamp replacement procedure. The UV controller 330 may store a previous state of the switch (i.e., on, off, open, or closed) and if the stored state is changed, the UV controller 330 may turn off the indicator light 340 and reset the lamp life timer (e.g., for another predetermined amount of time).

In other words, the UV controller 330 may include a lamp life timer configured to track an active operation time for the UV lamp 334 and the lamp fixture 320 may include the indicator light 340 configured to activate when the active operation time exceeds a predetermined threshold. Because the UV controller 330 may monitor the state of the clock reset switch 342, power doesn't have to be supplied to the lamp to inform a processor of the UV controller 330 that the UV lamp 334 has been changed. The clock reset switch 342 may be located in or near the UV lamp housing 324 (e.g., remote from the UV controller 330). The clock reset switch 342 leads may be isolated to provide both user shock protection as well as noise immunity.

The switched line 326 from the switch 310 may be electrically connected to the UV controller 330 and may thus enable the switch 310 to drive the UV controller 330. The UV controller 330 may in turn, drive the ballast 332, and thus, the UV lamp 334. Additionally, the UV controller 330 may drive the fan 336, which may feed air to be disinfected through an enclosed portion of the UV disinfection lamp, thereby providing disinfected air as an output.

The UV lamp 334, the UV controller 330, the fan 336, and the LEDs 322 may be connected to a neutral line 360. Thus, the circuit 300 for LED lighting with UV disinfection may apply UV lamp to air passed through the housing and thus, provide air and/or surface disinfection in fixture locations whose placement already exists in buildings.

The UV lamp 334 may be mounted on an interior of the lamp fixture 320 within an optically enclosed area. According to this aspect, the UV lamp 334 and the fan 336 may be contained within the housing and the fan 336 may blow air in a first side of the housing 324 and out of a second side of the housing 324 while the UV lamp 334 is on to engage in the air disinfection. According to another aspect, the fan 336 may pull air into the housing 324, which may be optically enclosed. Thus, the lamp fixture may include the fan 336 configured to blow air into the optically enclosed area of the housing 324. The UV controller 330 may be connected to the switched line 326 which may be connected to the switch 310.

When the switch 310 is switched on and UV air disinfection is selected from the switch, the switch 310 may provide a UV air disinfection signal across the switched line 326 to the UV controller 330 via one or more of the “place holder” bits of data transmitted from the switch 310 through the switched line 326 to the UV controller 330. The UV controller 330 may interpret this air disinfection signal as a command to activate the fan 336 and the UV lamp 334 contained within the housing 324.

According to other aspects, the UV lamp 334 may be mounted on an exterior facing portion of the lamp fixture 320. According to this aspect, the UV lamp 334 may mounted to an exterior portion of the housing 324 to engage in the surface disinfection while the UV lamp 334 is on. When the switch 310 is switched on and UV surface disinfection is selected from the switch, the switch may provide a UV surface disinfection signal across the switched line to the UV controller 330 via one or more of the “place holder” bits of data transmitted from the switch through the switched line to the UV controller 330.

The UV controller 330 may interpret this surface disinfection signal as a command to activate the UV lamp mounted exterior to the housing 324. In surface disinfection mode, one or more LEDs 322 or the indicator light 340 may be activated to indicate that the system is performing surface disinfection. In air disinfection mode, one or more LEDs 322 or the indicator light 340 may be activated in a different color or different flashing pattern to indicate that the system is performing air disinfection. The LEDs 322 or the indicator light 340 may be set to different colors or to flash to indicate different settings or messages depending on the system mode. For example, the LEDs 322 or the indicator light 340 may be set to red when the exterior UV lamp is active and white for when the UV lamp 334 needs to be replaced.

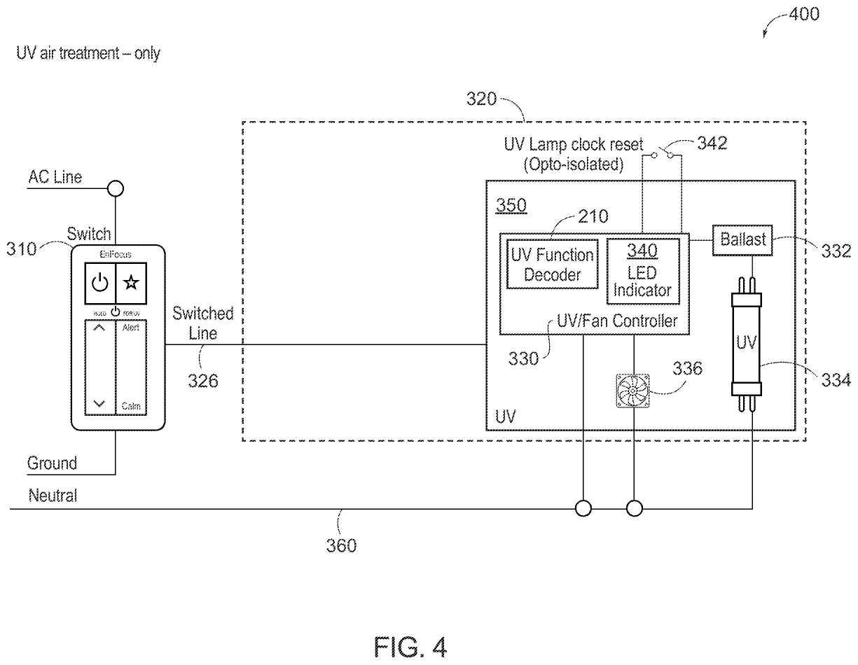

FIG. 4 is an exemplary component diagram of a circuit for light emitting diode (LED) lighting with ultraviolet (UV) disinfection, according to one aspect. FIG. 4 is an exemplary embodiment where the LEDs of FIG. 3 are removed, and thus, operation is merely focused on the UV disinfection portion of the circuit, without the LED lighting function.

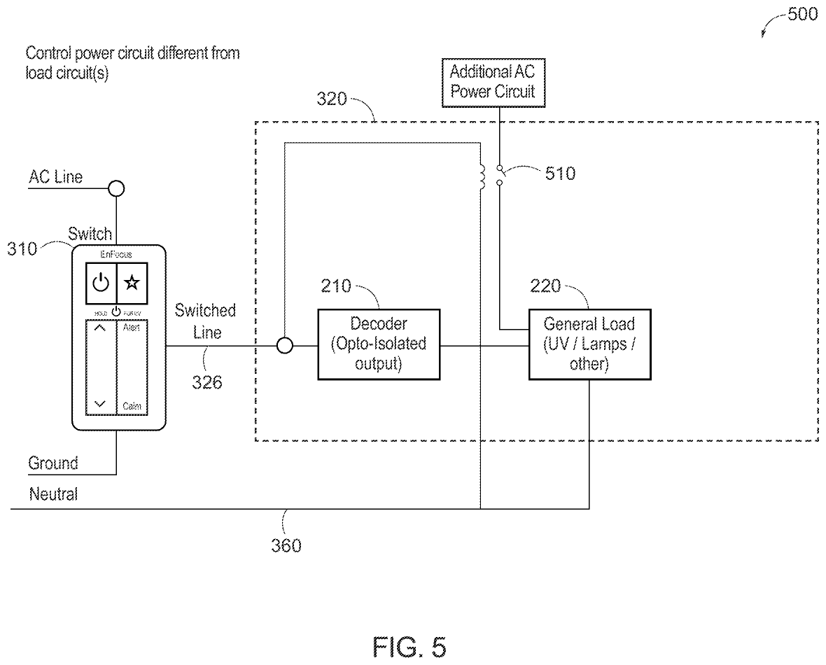

FIG. 5 is an exemplary component diagram of a circuit for light emitting diode (LED) lighting with ultraviolet (UV) disinfection, according to one aspect. According to one aspect, the decoder 210 may have an opto-isolated output, rather than being incorporated within the UV controller 330. In this way, the decoder 210 may be separated from the load, according to this aspect. In this regard, the circuit may include the switch 310 and a lamp fixture 320. The lamp fixture 320 may include an opto-isolated output decoder 210, one or more LEDs 322, and one or more UV disinfection lamps. An output signal from the switch may be fed, by a switched line to the opto-isolated output decoder and to an additional AC power circuit.

An output of the opto-isolated output decoder may be fed to one or more of the LEDs and/or one or more of the UV disinfection lamps 350. Thus, the control function power circuit may be separated from the operating power circuit. This allows the control of high current loads without having to size the control circuit transistors to handle the high currents. A relay 510 shown in FIG. 5 is a safety feature that prevents connection of the high current load in the absence of drive voltage from the switched load which guarantees that the load will not be engaged unless the UV controller voltage is present.

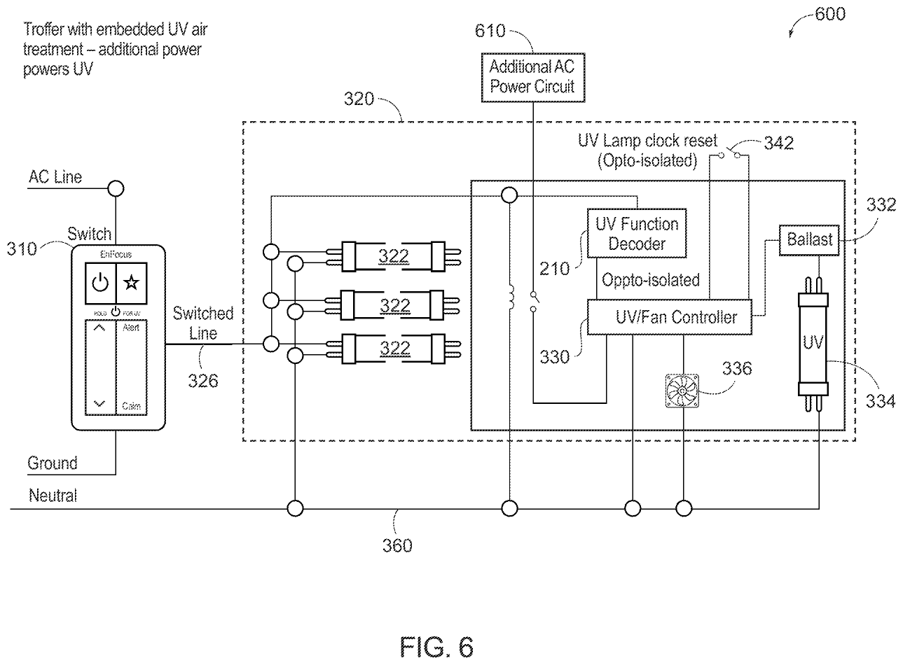

FIG. 6 is an exemplary component diagram of a circuit for light emitting diode (LED) lighting with ultraviolet (UV) disinfection, according to one aspect, using the concepts demonstrated in FIG. 5.

As seen in FIG. 6, a system for LED lighting with UV disinfection may include a switch 310 and a lamp or lamp fixture 320 equipped with one or more LEDs 322 and one or more UV disinfection lamps 350. The LED lamps 322 may be electrically connected to a switched line 326 which is connected to the switch 310. The UV disinfection lamp 350 may include a UV controller 330, a decoder 210, a ballast 332, a UV lamp 334, and a fan 336.

The decoder 210 may be a line decoder which may interpret brief line interruptions as commands to enable the lamp, disable the lamp, and/or change one or more settings associated with the lamp fixture 320. For example, a “place holder” bit location from data transmitted from the switched line may include UV control information indicative of a command for the UV controller 330 to control the UV lamp 334 and/or fan 336, such as but not limited to turning the UV lamp 334 on, turning the UV lamp 334 off, turning the fan 336 on, turning the fan 336 off, changing a speed of the fan 336, changing a UV level associated with the UV lamp 334, etc.

In this regard, the switch 310 may be configured to transmit a command to engage in a UV disinfection mode and the UV controller 330 may be configured to receive the command as a placeholder bit via a switched line 326. The UV controller 330 may be configured to receive the command to engage in the UV disinfection mode from the switch 310 via the switched line 326. The UV controller 330 may activate the UV lamp 334 based on the received command to engage in the UV disinfection mode.

Unlike FIG. 3, the LED lighting load current of FIG. 6 uses the control circuit while the UV load (e.g., the UV lamp) uses a separate circuit source (e.g., an additional AC power circuit 610). This enables implementation of higher power UV disinfection systems in buildings.

The UV controller may include a clock reset switch 342 which, when closed, resets a clock count on the UV lamp usage associated with replacing the UV lamp 334, which may be replaced on a periodic basis. The UV controller 330 may measure and/or track an amount of on time for the UV lamp 334. When a predetermined time (e.g., tracking the amount of on time for the UV lamp) has elapsed (e.g., 10,000 hours) one of the LED lamps 322 or an indicator light 340 may be lit to alert a user that it is time to replace the UV lamp 334. The clock reset switch 342 may be a single-pull-single-throw (SPST) type switch.

The user may change a state of the clock reset switch 342 (i.e., from open to closed or from closed to open) as part of the UV lamp replacement procedure. The UV controller 330 may store a previous state of the switch (i.e., on, off, open, or closed) and if the stored state is changed, the UV controller 330 may turn off the indicator light 340 and reset the lamp life timer (e.g., for another predetermined amount of time).

Because the UV controller 330 may monitor the state of the clock reset switch, power doesn't have to be supplied to the lamp to inform the processor of the UV controller 330 that the lamp has been changed. The clock reset switch 342 may be located in or near the UV lamp housing 324 (e.g., remote from the UV controller). The clock reset switch 342 leads may be isolated to provide both user shock protection as well as noise immunity.

The switched line 326 from the switch 310 may be electrically connected to the UV controller 330 and may thus enable the switch 310 to drive the UV controller 330. The UV controller 330 may in turn, drive the ballast 332, and thus, the UV lamp 334. Additionally, the UV controller 330 may drive the fan 336, which may feed air to be disinfected through an enclosed portion of the UV disinfection lamp, thereby providing disinfected air as an output. The UV lamp 334, the UV controller 330, the fan 336, and the LEDs 322 may be connected to a neutral line 360.

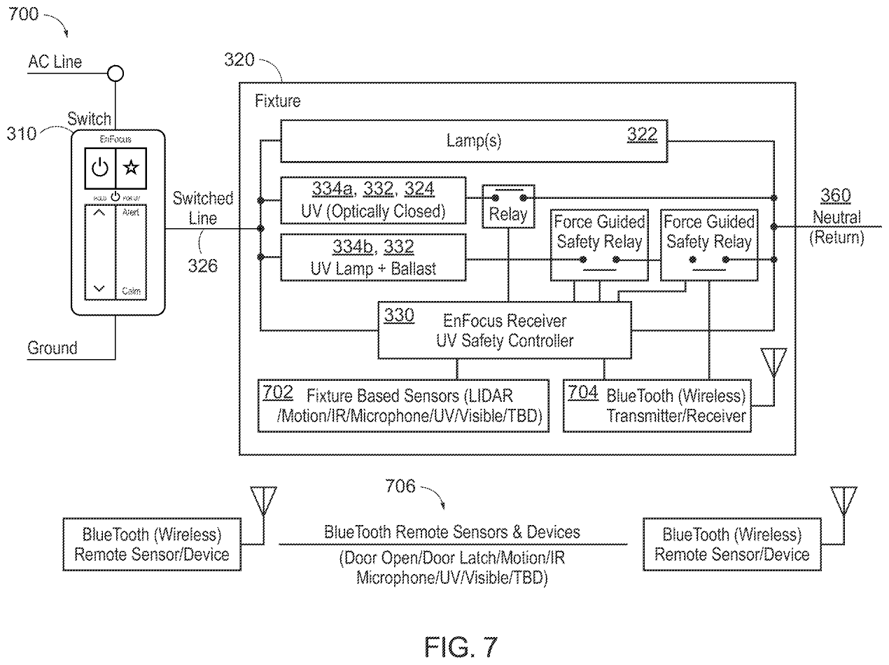

FIG. 7 is an exemplary component diagram of a circuit for light emitting diode (LED) lighting with ultraviolet (UV) disinfection, according to one aspect. A UV lamp 334b may be mounted on an exterior of the lamp fixture 320. Another UV lamp 334a may be mounted on an interior of the lamp fixture 320 within an optically enclosed area and the lamp fixture may include a fan configured to blow air into the optically enclosed area.

The lamp fixture 320 of FIG. 7 includes the UV controller 330, and the UV controller 330 may be in communication with one or more mounted sensors 702 (e.g., mounted to the lamp fixture) and a communication interface 704. The communication interface 704 may be configured to receive sensor data from a remote sensor 706. It will be appreciated that the sensor 702 may be remote (i.e., not mounted to the fixture) without departing from the scope of the disclosure.

The sensors 702, 706 may be of any known sensor type, including, but not limited to occupancy sensors, radar sensors, heartbeat sensor, LIDAR sensors, motion detector sensors, microphones, UV sensors, infrared sensors, visible light sensors, proximity sensor, door sensor, etc. These sensors 702, 706 may detect the presence of humans within a room where the circuit for LED lighting with UV disinfection is equipped.

The communication interface 704 may follow Bluetooth® protocols, and may include a transmitter, a receiver, a transceiver, etc. The communication interface 704 may be in communication with one or more remote sensors 706 and/or remote devices, which may include similar sensors as the sensors in communication with the UV controller 330, and may be configured to detect the presence of humans within a room where the circuit for LED lighting with UV disinfection is equipped.

Regardless of which set of sensors (e.g., the remote sensors 706 or the sensors 702 attached to the lamp) are utilized, when the presence of humans is detected, the UV controller 330 may disable the UV lamp mounted exterior to the lamp. In this way, the UV controller 330 may deactivate the UV lamp based on the data from the remote sensor being indicative of a detected presence of a life form. Thus the UV controller 330 may deactivate the UV lamp based on data from the mounted sensor being indicative of a detected presence of a life form.

According to one aspect, the optically enclosed UV lamp may continue to operate in this scenario. According to another aspect, the optically enclosed UV lamp may be deactivated based on the data from the remote sensor being indicative of a detected presence of a life form.

While the embodiments shown in FIGS. 1-7 illustrate aspects where no neutral is present at the switch housing, it will be appreciated that the embodiments shown in FIGS. 1-7 are also effective when the neutral is available at the switch (i.e., a 3-wire switch configuration).

It will further be appreciated that various of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A system for ultraviolet (UV) disinfection, comprising:
a switch configured to transmit a command to engage in a UV disinfection mode;
a lamp fixture including:
a UV lamp; and
a UV controller,
wherein the UV controller is configured to receive the command to engage in the UV disinfection mode from the switch via a switched line, and
wherein the UV controller activates the UV lamp based on the received command to engage in the UV disinfection mode.

2. The system for ultraviolet (UV) disinfection of claim 1, the lamp fixture further including at least one LED lamp for general lighting purposes, wherein the at least one LED lamp is controlled via the switched line by the switch that also commands the UV controller.

3. The system for ultraviolet (UV) disinfection of claim 1, wherein the UV lamp is mounted on an interior of the lamp fixture within an optically enclosed area and the lamp fixture includes a fan configured to blow air into the optically enclosed area.

4. The system for ultraviolet (UV) disinfection of claim 1, wherein the lamp fixture includes an indicator light configured to activate when the lamp fixture is engaged in the UV disinfection mode.

5. The system for ultraviolet (UV) disinfection of claim 1, wherein the lamp fixture includes a communication interface configured to receive sensor data from a remote sensor.

6. The system for ultraviolet (UV) disinfection of claim 5, wherein the UV controller deactivates the UV lamp based on the data from the remote sensor being indicative of a detected presence of a life form.

7. The system for ultraviolet (UV) disinfection of claim 1, wherein the UV controller includes a lamp life timer configured to track an active operation time for the UV lamp.

8. The system for ultraviolet (UV) disinfection of claim 7, wherein the lamp fixture includes an indicator light configured to activate when the active operation time exceeds a predetermined threshold.

9. The system for ultraviolet (UV) disinfection of claim 1, wherein the UV controller is configured to receive the command as a placeholder bit via the switched line.

10. The system for ultraviolet (UV) disinfection of claim 1, wherein the lamp fixture includes a mounted sensor and wherein the UV controller deactivates the UV lamp based on data from the mounted sensor being indicative of a detected presence of a life form.

11. A system for ultraviolet (UV) disinfection, comprising:
a switch configured to transmit a command to engage in a UV disinfection mode;
a lamp fixture including:
a first UV lamp mounted on an exterior of the lamp fixture;
a second UV lamp mounted on an interior of the lamp fixture within an optically enclosed area;
a fan configured to blow air into the optically enclosed area; and
a UV controller,
wherein the UV controller is configured to receive the command to engage in the UV disinfection mode from the switch via a switched line, and
wherein the UV controller activates the UV lamp based on the received command to engage in the UV disinfection mode.

12. The system for ultraviolet (UV) disinfection of claim 11, wherein the switch transmits at least one command to the lamp fixture by providing brief interruptions of the power line to the lamp fixture, wherein the lamp fixture includes a decoder which interprets and executes the at least one command.

13. The system for ultraviolet (UV) disinfection of claim 11, wherein the lamp fixture includes a communication interface configured to receive sensor data from a remote sensor.

14. The system for ultraviolet (UV) disinfection of claim 13, wherein the UV controller deactivates the first UV lamp based on the data from the remote sensor being indicative of a detected presence of a life form.

15. A system for ultraviolet (UV) disinfection, comprising:
a switch configured to transmit a command to engage in a UV disinfection mode;
a lamp fixture including:
a UV lamp mounted on an interior of the lamp fixture within an optically enclosed area;
a fan configured to blow air into the optically enclosed area; and
a UV controller,
wherein the UV controller is configured to receive the command to engage in the UV disinfection mode from the switch via a switched line, and
wherein the UV controller activates the UV lamp based on the received command to engage in the UV disinfection mode.

16. The system for ultraviolet (UV) disinfection of claim 15, wherein the lamp fixture includes an indicator light configured to activate when the lamp fixture is engaged in the UV disinfection mode.

17. The system for ultraviolet (UV) disinfection of claim 15, wherein the lamp fixture includes a communication interface configured to receive sensor data from a remote sensor.

18. The system for ultraviolet (UV) disinfection of claim 17, wherein the UV controller deactivates the UV lamp based on the data from the remote sensor being indicative of a detected presence of a life form.

19. The system for ultraviolet (UV) disinfection of claim 15, wherein the UV controller is configured to receive the command as a placeholder bit via the switched line.

20. The system for ultraviolet (UV) disinfection of claim 15, wherein the lamp fixture includes a mounted sensor and wherein the UV controller deactivates the UV lamp based on data from the mounted sensor being indicative of a detected presence of a life form.
